(19) United States
(12) Patent Application Publication
(10) Pub. No.: US 2019/0255304 A1
(43) Pub. Date: Aug. 22, 2019

(54) HANDS-FREE MEDICAMENT APPLICATOR AND METHODS OF USING SAME

(71) Applicant: Steven Lemberger, Freehold, NJ (US)

(72) Inventor: Steven Lemberger, Freehold, NJ (US)

(21) Appl. No.: 16/269,224

(22) Filed: Feb. 6, 2019

Related U.S. Application Data
(60) Provisional application No. 62/631,716, filed on Feb. 17, 2018.

(51) Int. Cl. A61M 35/00; A45D 40/26

(52) U.S. Cl. CPC A61M 35/003; A61K 9/0014; A45D 40/26

(57) ABSTRACT

A substance applicator device having a proximal end and a distal end includes a wedge-shaped rigid housing having a lower surface and an upper surface, the upper surface defining a cavity, a fixed valve seat disposed within the rigid housing and defining a chamber therein, a pump disposed on the valve seat and configured to draw the substance from the chamber, and a toggle disposed in the cavity of the rigid housing and moveable relative to the housing, the toggle being in contact with the pump, the toggle including an upper cover, a lower frame and a well disposed therebetween, the upper cover having at least one hole and the lower frame having at least one aperture.

### CROSS-REFERENCE TO RELATED APPLICATIONS

This application claims priority to U.S. Provisional Application Ser. No. 62/631,716, entitled “HANDS-FREE MEDICAMENT APPLICATOR AND METHODS OF USING SAME”, filed on Feb. 17, 2018, the contents of which is hereby incorporated by reference herein as if fully set forth herein.

### FIELD OF THE DISCLOSURE

The present disclosure relates to medicament applicators. More particularly the present disclosure relates to applicators capable of supplying a cream, ointment, medication or other substance to a patient's foot.

### BACKGROUND OF THE DISCLOSURE

Podiatric patients often need to apply a substance to their feet, ankles, and digits as part of a treatment regimen. For example, a physician may prescribe daily inspection and application of a topical medication or substance as a remedy to bacterial infections, ulcers, cuts, sores, and/or other ailments or abnormalities. Many patients are physically unable to follow their physician's advice due to their physical condition. For example, older or obese patients may have trouble reaching their feet with their hands to apply the treatment. Other individuals may be unable to reach or bend either their backs, arms, waist, or knees due to injury. Yet others may simply dislike the idea of touching their feet, especially if diseased.

Thus, it would be desirable to have a device and a method of applying a substance to the feet without having to reach with the hand or bend at the back. Such a device may be useful for older or obese patients as well as other patients who have physical or mental limitations that prevent them from touching their feet with their hands.

### SUMMARY OF THE DISCLOSURE

In some embodiments, a substance applicator device having a proximal end and a distal end includes a rigid housing, a valve seat disposed within the rigid housing and defining a chamber therein, a pump disposed on the valve seat, and a toggle projecting from the rigid housing and moveable relative thereto, the toggle being in contact with the pump.

In some embodiments, a substance applicator device having a proximal end and a distal end includes a wedge-shaped rigid housing having a lower surface and an upper surface, the upper surface defining a cavity, a fixed valve seat disposed within the rigid housing and defining a chamber therein, a pump disposed on the valve seat and configured to draw the substance from the chamber, and a toggle disposed in the cavity of the rigid housing and moveable relative to the housing, the toggle being in contact with the pump, the toggle including an upper cover, a lower frame and a well disposed therebetween, the upper cover having at least one hole and the lower frame having at least one aperture.

### BRIEF DESCRIPTION OF THE DISCLOSURE

Various embodiments of the presently disclosed applicators are shown herein with reference to the drawings, wherein:

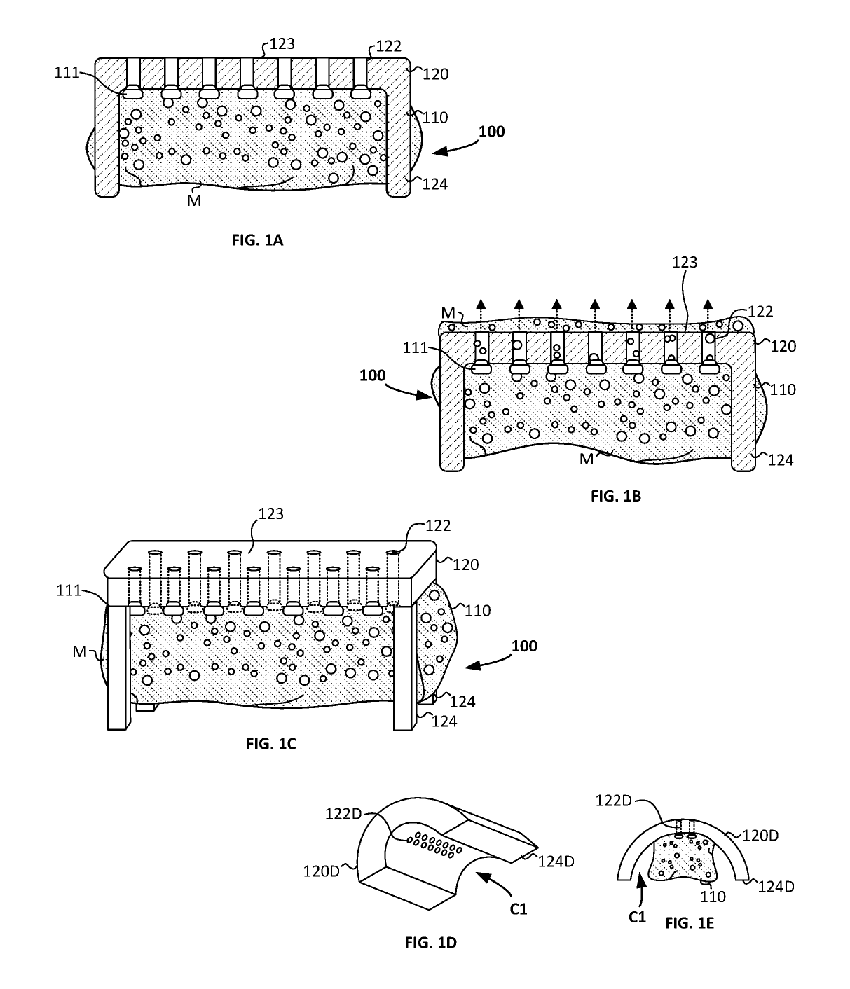

FIGS. 1A-C are schematic side and perspective views of an applicator device according to one embodiment;

FIGS. 1D-E are schematic perspective and cross-sectional views of another embodiment of a platform;

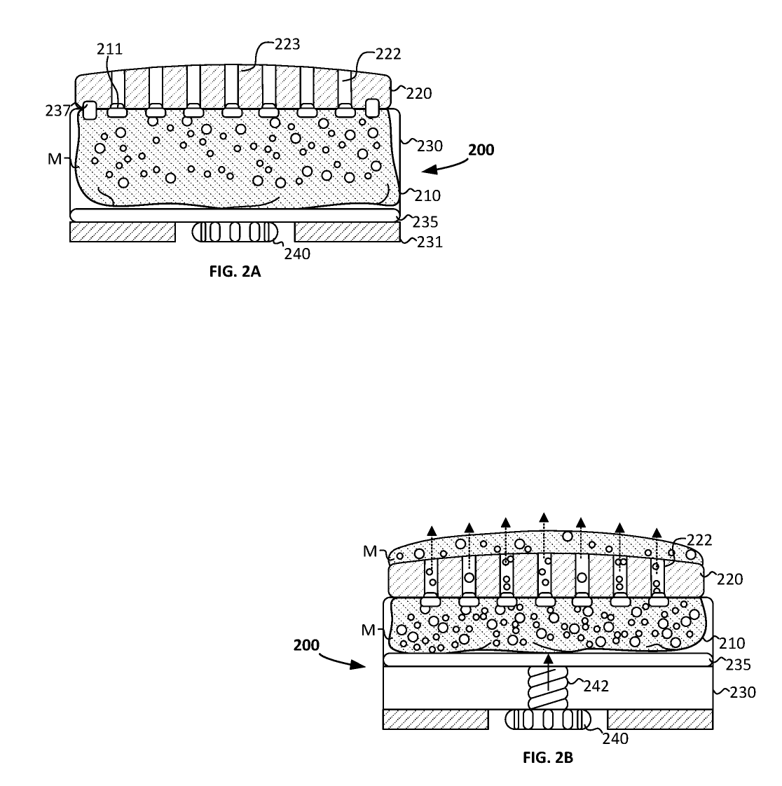

FIGS. 2A-B are schematic side views of an applicator device having a container according to another embodiment;

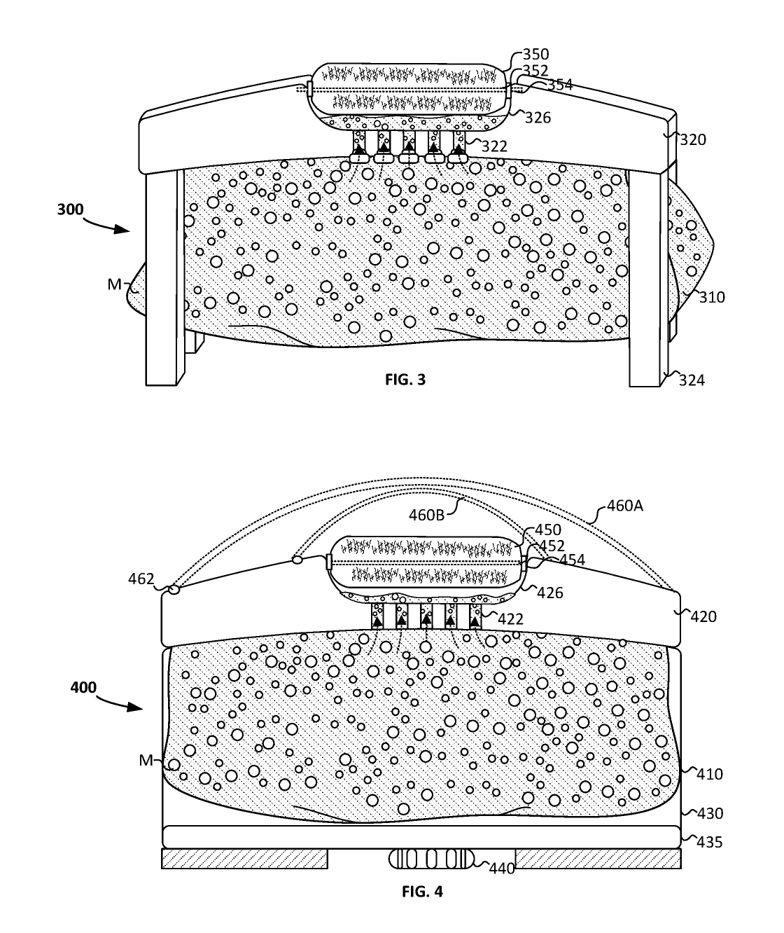

FIG. 3 is a schematic perspective view of an applicator device having a roller according to another embodiment;

FIG. 4 is a schematic side view of an applicator device having a roller according to another embodiment;

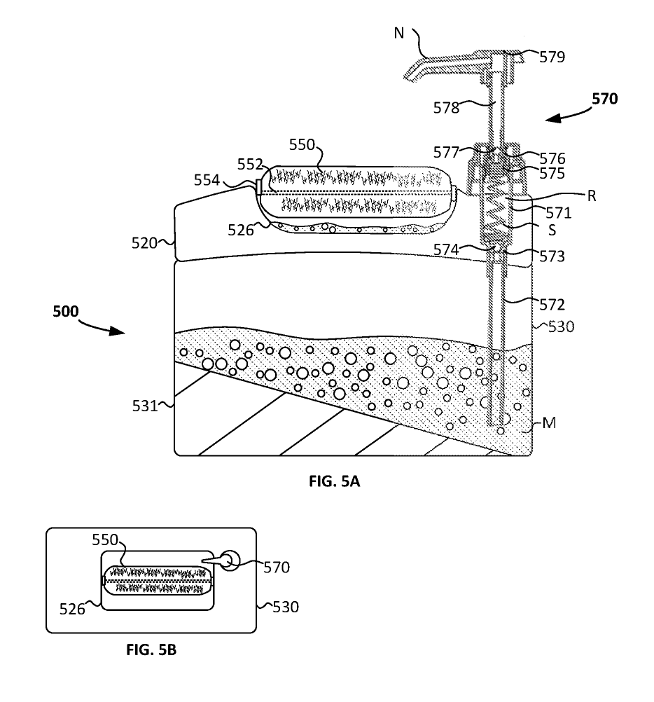

FIGS. 5A-B are schematic side and top views of an applicator device having a pump according to one embodiment;

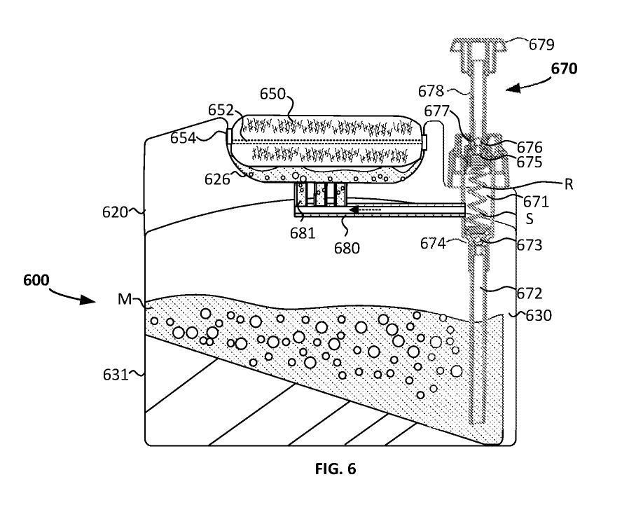

FIG. 6 is a schematic side view of an applicator device having a pump according to another embodiment;

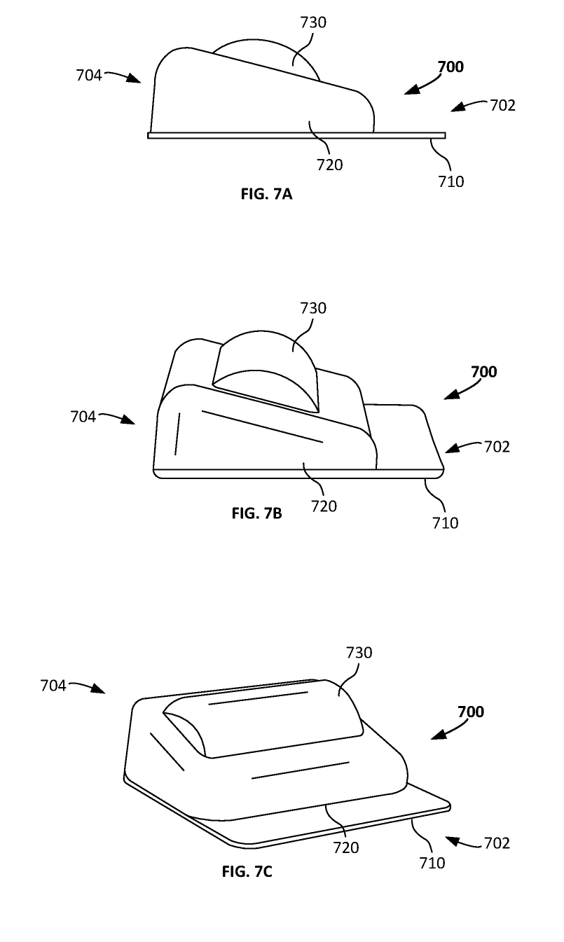

FIGS. 7A-C are schematic side and perspective views of an applicator device according to another embodiment of the present disclosure;

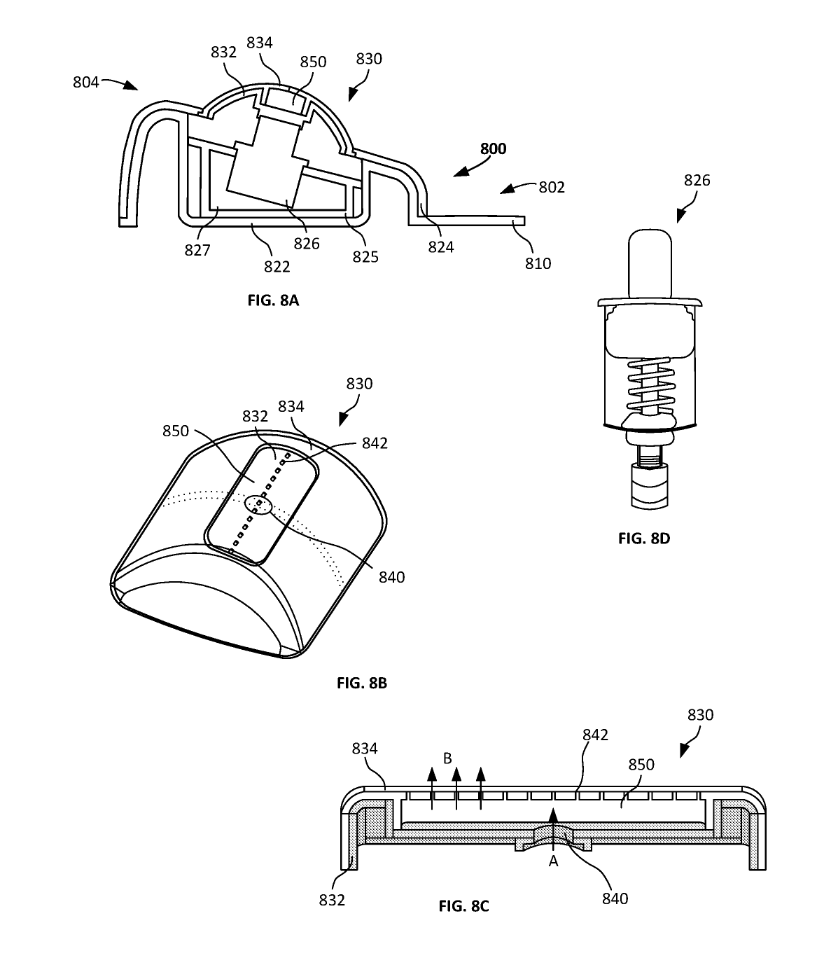

FIGS. 8A-D are a schematic cross-sectional view of an applicator, a detailed view of a toggle and a cross-sectional view of same, and a detailed view of a pump, respectively; and

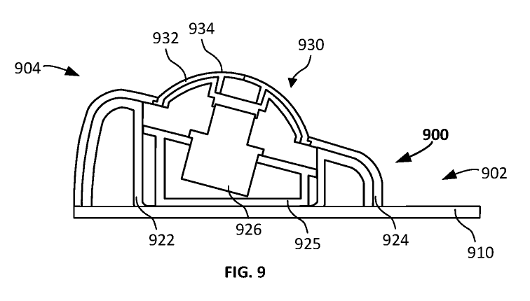

FIG. 9 is another example of an applicator having an integrated base and housing.

Various embodiments of the present invention will now be described with reference to the appended drawings. It is to be appreciated that these drawings depict only some embodiments of the disclosure and are therefore not to be considered limiting of its scope.

### DETAILED DESCRIPTION

Despite the various improvements that have been made to medicament applicators, conventional methods suffer from some shortcomings as discussed above.

There therefore is a need for further improvements to the devices and methods of storing and applying medicaments to patients' feet. Among other advantages, the present disclosure may address one or more of these needs.

FIG. 1 illustrates a first embodiment of an applicator 100 that generally includes a pouch 110 and a platform 120. Pouch 110 may define a reservoir adapted to contain a gel, semi-solid, or liquid product therein, such as a cream, a lotion, a foam, an ointment, a topical medicament, a gel, a soap, or other suitable product. The product may be homogenous or may formed of a first group of material and a second group of material (e.g., a scrub, an exfoliant, and/or a bead) being dispersed within the first group of material. As used herein, the term “medicament” is used generally as a placeholder for any of the substances described above, but it will be understood that any of the substances may be used in any of the embodiments (or combination of embodiments) described herein.

In some examples, pouch 110 may include a plastic material, or a polymeric material, rubber, a metal or combinations thereof. Pouch 110 may be sized to retain approximately 3 oz to 40 oz of a medicament “M” shown in the figures as a gray substance having circular particles (e.g., an exfoliant), and may include one or more valves 111 for dispensing the medicament from the inside of the pouch to the outside.

As shown, applicator 100 may also include a rigid platform 120 made of a generally rectangular member having a number of passageways being formed therethrough from a lower surface to an upper surface. The platform may be formed of a hard plastic, a metal, a wood, a rubber, or any other suitable material. The number and configuration of valves 111 of pouch 110 may be complementary to the number and configuration of passageways 122 on platform 120. Thus, when a user squeezes pouch 110 as shown in FIG. 1B, medicament “M” may flow through valves 111 and passageways 122 to the top surface of platform 120 where it will be available for the patient. As shown in the perspective view of FIG. 1C, the number and spacing of passageways may be selected so that when the pouch 110 is squeezed, medicament “M” is evenly dispersed on the upper surface 123 of platform 120. Optionally, platform 120 may include one or more legs 124 extending from the rectangular portion. For example, platform 120 may include four legs 124 as shown, so that the platform is in the shape of a table, with the pouch being nested under the rectangular portion.

To use the applicator, the user or patient may squeeze the pouch 110 to release the medicament from inside the pouch to the top surface 123 of platform 120. The user may then gently place their foot on the platform, which sits on legs 124 and slide their foot over the platform 120 so that the medicament is applied to their foot.

Instead of a table-shaped platform, a convex platform may be utilized as shown in FIG. 1D. Convex platform 120D is generally shaped as half a tubular member, the platform having a number of passageways 122D from one side to the other. In this example, instead of legs, the platform 120D may be disposed flat on edges 124D so that a number of substantially semi-circular cross-sections are formed, the platform 120D being capable of accepting within its concavity “C1” a pouch (see FIG. 1E). It will be understood that any of the platforms discussed herein may be substituted for platforms of any of the other embodiments.

FIGS. 2-6 illustrate various embodiments that share similar elements with those of FIG. 1. Where the same terminology is used and like-numbered elements are found, it will be understood that any of the features described for the element may be equally applicable for the variant. For example, pouch 210 may be formed of the same materials and in the same configurations as pouch 110.

FIGS. 2A and 2B show a variant of the applicator of FIGS. 1A-C. In this embodiment, applicator 200 includes a pouch 210 similar to that of pouch 110, and a platform 220 that is similar to platform 120. As shown in FIGS. 2A and 2B, platform 220 may include a generally convex upper surface 223. Another difference from the first embodiment is that applicator 200 includes a container 230 formed of a plastic, a metal, a rubber, a wood, a polymeric material or other suitable material, or combinations of materials such as those described herein. The container 230 may house pouch 210, and include a notched base 231, a moveable member 235 that is mechanically coupled to knob 240 via threaded member 242, and a number of clips 237 to secure the container to the platform. Moveable member 235 may be configured to translate from a bottom surface of the container toward the platform as the user turns knob 240. As the user turns knob 240, moveable member 235 pushes pouch 110 upward toward platform 120 until the pouch begins to release medicament “M” through the valve 211, the passageways 222 to the upper surface of platform 220 for use by the patient. In this manner, a controlled release of the medicament is possible. Additionally, because container 230 is rigid in certain examples, and because knob 240 is disposed within the cavity of the notched base 231, the user may place the applicator flat on the ground and place their foot on the platform without fear of discharging the entire contents of the pouch. The medicament may be delivered to upper surfaces 223 and the device may be placed on the floor where the patient steps on the device and receives the medicament on their feet.

FIG. 3 shows yet another embodiment of an applicator 300, which includes a pouch 310 having a medicament “M” and a curved platform 320. The main difference in this embodiment is that the top of platform 320 includes a recessed well 326 at the center of the platform and disposed directly over passageways 322 so that discharged medicament flows into the well as shown by the arrows. Platform 320 may include optional legs 324 as discussed above. Applicator 300 further includes a disbursing member in the form of a rotatable roller 350, the roller having a transverse rod 352 extending longitudinally therethrough, the rod being affixed on both sides to shoulders 354 of platform 320. As shown, the roller 350 may be at least partially disposed within the cavity that defines well 326, and partially elevated above the highest point of platform 320. The roller may be spaced from the bottom of the recessed wall by a distance of between 0.1 inches to 3 inches so that it is capable of rotating freely and contacts the medicament in the well.

In this embodiment, the user may squeeze the pouch 310 to introduce medicament “M” into the well 326. The user may then slide their foot over the roller 350, the roller being capable of absorbing medicament “M” from the well and transferring the medicament to the patient's foot as it rotates.

Another variation is shown in FIG. 4, in which applicator 400 includes a pouch 410 having a medicament “M” and a curved platform 420. In this embodiment, pouch 410 is housed within rigid container 430, the container having a moveable member 435 and a knob 440 similar to those found in the embodiment of FIG. 2, the knob being actuatable for controlled released of the medicament. In some examples, a pouch 410 is not used, and the medicament is disposed directly within the container 430. Platform 420 also includes a number of passageways 422 and a recessed well 426 similar to those of FIG. 3. Additionally, a protective cover 460A may be disposed over roller 450 and well 426 to keep the roller sanitary and/or to prevent the roller from drying out after use. As shown, cover 460A may be large enough to protect almost all of platform 420, or a smaller cover 460B may be used that protects only the roller and the well. Either covering may be affixed to the container in various methods, and removably disposed to reveal the roller such as by using a hinge 462 on one side of the cover. Again, container 230 may be rigid so that the patient may step on the device and slide their feet across the roller to receive the medicament.

Instead of manually turning a knob at the bottom of a container to release medicament “M” from the interior of the container, a pumping mechanism may be used as will be described with reference to FIGS. 5 and 6.

As shown, in applicator 500, a container 530 includes a predetermined amount of medicament “M”. The applicator 500 also includes a platform 520 having a recessed well 526 and a roller 550 extending over a rod 552 that extends between shoulder 554. In this example, a pouch is optional, and the medicament is disposed directly within container 530, the container having an inclined floor 531 that extends diagonally across the interior of the container as shown so that the medicament naturally flows down to the incline toward the pump mechanism.

Applicator 500 further includes pumping mechanism 570 at least partially disposed within container 530 and to one side of the container at the lower end of inclined floor 531, the pumping mechanism being capable of transferring medicament from the interior of the container to the recessed well 526. Pumping mechanism may be similar to certain soap dispensers. In one example, a typical pumping mechanism 570 mainly includes a hollow cylindrical member 571 in communication with a diptube 572 disposed adjacent the lower end of the inclined floor 531, the cylindrical chamber mechanism defining a reservoir “R” having at the lower portion thereof a valve seat 573, a ball 574, and a piston 575. A second upper valve seat 576, and a second upper ball 577 are disposed above a compression spring “S” mounted between valve seat 573 and piston 575. A hollow operating rod 578 is mounted above the piston, and in communication with an interior of a pressing member 579 and a nozzle “N” so that when pressing, member 579 and operating rod 578 are pushed downward by an external pressure, piston 575 is moved downward accordingly, and the air and/or medicament contained in reservoir “R” will push upward the ball 577 to thus discharge air and/or medicament through nozzle “N”. When external pressure pressing the pressing member and the rod is released, spring “S” pushes upward piston 575 to thus form a vacuum reservoir to in turn move the medicament from the container through diptube 572 into reservoir room “R”. If pressing member 579 is pushed downward again, the bottled medicament stored in the room will flow through the piston to be discharged out of the nozzle and into recessed well 526, where it is made available for absorption by roller 550 and ultimately applied to the patient's foot via roller 550.

FIG. 6 shows another example of an applicator 600 that is essentially the same as applicator 500 with minor variations. Like-numbered elements shown in FIG. 6 will be the same as those described in FIGS. 5A and 5B, except that they will be preceded with a “6” instead of a “5”. For example, container 530 of FIG. 5 will be essentially the same as container 630 with few variations, and any differences between the two embodiments will be explained.

As shown, container 630 includes a predetermined amount of medicament “M”. The applicator 600 also includes a platform 620 having a recessed well 626 and a roller 650 extending over a rod 652 that extends between shoulders 654 as previously described. In this example, a pouch is not used, and the medicament is disposed directly within container 630, the container having an inclined floor 631.

Applicator 600 includes a pumping mechanism 670 that is similar to pumping mechanism 570 of FIGS. 5A-B, with two main exceptions. First, pumping mechanism 670 does not include a nozzle “N” as previously described, but instead terminates in a pressing member 679. Instead of a nozzle, pumping mechanism 670 includes a side branch 680 in communication with reservoir room “R”, the branch being hollow so that medicament may flow therethrough and into the recessed well 626 via one or more channels 681. Thus, in this embodiment, medicament “M” flows into the recessed well from beneath the well instead of being pumped out of nozzle at the top of the device into the well. In use, the patient may use the pumping mechanism to draw medicament from the container, into the pump, through the branch 680, the channels 681 and into the recessed well 626. After sufficient medicament has been introduced into the well, the patient may then sit in a chair and slide their foot over the roller to apply the medicament onto their foot, the roller being capable of absorbing or picking up the substance and evenly applying it to the patient's foot.

FIGS. 7A-C are schematic side and perspective views of another applicator device 700 similar to those previously described. It will be understood that any of the features described above may be combinable with applicator device 700. Generally, applicator device 70 extends between a proximal end 702 and a distal end 704, and includes a flat base 710, a wedge-shaped plastic or metallic rigid, housing 720 and a toggle 730 projecting out of the housing. Toggle 730 may be ergonomically-shaped to have a curvature so that a user can step or press on it with their feet. More of the details of applicator 700 are shown with references to FIGS. 8A-D. Where the same terminology is used and like-numbered elements are found, it will be understood that any of the features described for the element may be equally applicable for the variant.

The cross-sectional view of FIG. 8A shows one example of a wedge-shaped applicator 800, which extends between proximal end 802 and distal end 804. Applicator 800 includes a unitary stabilizing base 810 extending from the lower housing 822, and an upper housing 824 covering parts of the lower housing. Specifically, lower housing 822 extends away from the device to increase its footprint and form the stabilizing base 810. In another example, shown in FIG. 9, base 910 and lower housing 922 are separately formed. Turning back to FIG. 8A, an upper housing 824 covers portions of the lower housing 822 and provides a compartment for receiving a valve seat 825 defining a reservoir or chamber 827. Valve seat 825 may be fixed within the housing and incapable of moving relative thereto. Valve seat 825 may also include an inclined upper surface parallel with the upper surface of the upper housing and a compressible pump 826 positioned on the valve seat 825, and arranged at an angle (e.g., substantially orthogonal) to the valve seat's upper surface, the pump being in communication with the chamber at one end and in contact with a toggle. Toggle 830 projects from the wedge-shaped housing, and may be moveable relative to the housing to compress the pump, the toggle having a lower frame 832 and an upper cover 834, and a well 850 defined therebetween.

Details of toggle 830 will be appreciated by examining the perspective and cross-sectional views of the toggle, shown in FIGS. 8B-C. As shown in these figures, toggle 830 generally includes a supporting lower frame 832 and a semi-cylindrical cover 834 (frame being shaded in gray in the cross-sectional view). Frame 832 may define a central aperture 840 that provides communication between the pump 826 and well 850 so that a medicament is capable of exiting the pump and entering the well in the direction of arrow “A”. Pump 826 may be spring-loaded as shown in FIG. 8D, the pump being capable of being compressed to a first condition to draw a substance up through the pump, and the spring of the pump allowing the pump to return to its extended condition when no external force is applied thereto.

A row of pathways or holes 842 is formed in cover 834 so that the medicament may pass from the well 850 to the top surface of the toggle to contact the patient's skin as shown by arrows “B”. As shown, cover 834 includes 13 holes aligned across the longitudinal axis of the cover. It will be understood that any number of holes (e.g., a single hole, two, three, four, or more) may be formed in the cover, and that the size, shape and/or arrangement of the holes may be adjusted based on the type or viscosity of the medicament. In at least some examples, cover 834 is translucent so that the contents of the well is clearly visible to the patient. Additionally, frame 832 may be in contact with pump 826 so that by pressing on cover 834, frame 832 is moved downward to depress pump 826 to draw medicament from chamber 827 to well 850 via central aperture 840. When enough medicament has filled the well, it will exit through holes 842 to the surface of toggle 830 and be made available to contact the patient's skin (e.g., on the patient's foot). In at least some examples, toggle 830 including the lower frame and the cover may be removable, as well as the pump. In such examples, chamber 827 may be refillable with medicament when the previous amount has been exhausted. Additionally, it will be appreciated that instead of placing the medicament directly into the chamber 827, that a pouch of medicament may be disposed within the chamber, the pouch being coupled to a lower end of pump 826 and having an opening in communication with the pump so that the pump is capable of drawing the medicament out of the pouch and into well 850.

In use, the patient may fill chamber 827 with a medicament (e.g., a cream) and set the applicator on the floor. The patient my then press with their foot on the toggle 830 one or more times. Pressing of the toggle acts to bring cream from the chamber (or form the pouch within the chamber) to the well via the central aperture 840. As the well fills it may overflow and exit through the holes, where it will be applied to the patient's foot without requiring the patient to reach with their hands. The base may stabilize the assembly and prevent it from tipping.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. A substance applicator device having a proximal end and a distal end, comprising:
a rigid housing;
a valve seat disposed within the rigid housing and defining a chamber therein;
a pump disposed on the valve seat; and
a toggle projecting from the rigid housing and moveable relative thereto, the toggle being in contact with the pump and capable of drawing a substance from the chamber into the toggle.

2. The substance applicator device of claim 1, wherein the rigid housing includes an upper housing and a lower housing.

3. The substance applicator device of claim 1, wherein the rigid housing is wedge-shaped.

4. The substance applicator device of claim 1, further comprising a flat stabilizing base at the proximal end of the applicator device, the stabilizing base extending beyond the housing away from the pump.

5. The substance applicator device of claim 2, further comprising a stabilizing base integral with the lower housing.

6. The substance applicator device of claim 1, wherein the toggle includes an upper cover and a lower frame.

7. The substance applicator device of claim 6, wherein the lower frame includes a central aperture.

8. The substance applicator device of claim 6, wherein the upper cover includes a plurality of holes longitudinally aligned along the upper cover.

9. The substance applicator device of claim 6, wherein the upper cover is translucent.

10. The substance applicator device of claim 6, wherein the lower frame and the upper cover define a well therebetween, and the pump is capable of drawing a substance from the chamber to the well.

11. The substance applicator device of claim 1, wherein the pump is compressible and includes a spring.

12. The substance applicator device of claim 1, further comprising a substance disposed within the chamber.

13. The substance applicator device of claim 12, wherein the substance is a medicament.

14. The substance applicator device of claim 12, wherein the substance is a cream.

15. A substance applicator device having a proximal end and a distal end, comprising:
a wedge-shaped rigid housing having a lower surface and an upper surface, the upper surface defining a cavity;
a fixed valve seat disposed within the rigid housing and defining a chamber therein;
a pump disposed on the valve seat and configured to draw a substance from the chamber; and
a toggle disposed in the cavity of the rigid housing and moveable relative to the housing, the toggle being in contact with the pump, the toggle including an upper cover, a lower frame and a well disposed therebetween, the upper cover having at least one hole and the lower frame having at least one aperture.

16. The substance applicator device of claim 15, wherein the pump is compressible and includes a spring.

17. The substance applicator device of claim 15, wherein the at least one hole includes a plurality of holes.

18. The substance applicator device of claim 15, wherein the upper cover is translucent.

19. The substance applicator device of claim 15, further comprising a stabilizing base at the proximal end of the applicator device, the stabilizing base extending beyond the rigid housing away from the pump.

20. The substance applicator device of claim 15, further comprising a substance disposed within the chamber.
